(19) United States
(12) Patent Application Publication
(10) Pub. No.: US 2008/0281284 A1
(43) Pub. Date: Nov. 13, 2008

(54) FLUID COLLECTION SYSTEM

(76) Inventors: Michael H. Garfield, Oviedo, FL (US); Paul Hendrixson, Cumming, GA (US)

Correspondence Address: GRAY ROBINSON, P.A., P.O. Box 2328, FT. LAUDERDALE, FL, 33303-9998, US

(21) Appl. No.: 11/746,128

(22) Filed: May 9, 2007

Related U.S. Application Data
(63) Continuation-in-part of application No. 11/745,473, filed on May 8, 2007.

(51) Int. Cl. A61M 1/00

(52) U.S. Cl. 604/327

(57) ABSTRACT

A system is provided for collecting bodily fluids, and particularly urine from the bladder of a patient, comprising a catheter inserted into the bladder, a low pressure, cyclic magnetic valve coupled to the catheter, a collection bag, and, a quick-release coupler interposed between the magnetic valve and the collection bag.

### RELATED APPLICATION

This application is a continuation-in-part of U.S. patent application Ser. No. 11/745,473, filed May 8, 2007 and entitled “COUPLER.”

### FIELD OF THE INVENTION

This invention relates to medical devices, and, more particularly, to a system for the collection and disposal of bodily fluids following surgery or in patients with certain medical conditions.

### BACKGROUND OF THE INVENTION

Normal bladder function includes cyclic filling of the bladder with urine and periodic voiding when pressure buildup reaches a noticeable level. The bladder detrusor muscle controls voiding of the bladder and must be exercised like any other muscle to maintain healthy tone. When healthy, the bladder periodically empties or washes out, allowing bacteria and other potentially harmful micro-organisms to exit the body thus preventing infection.

A variety of medical conditions can interfere with the normal function of the bladder and the bladder detrusor muscle. When patients have difficulty urinating, a Foley catheter or other indwelling catheter is usually employed to open the bladder and allow voiding of urine. In typical applications, the catheter is coupled to a collection bag, either directly or through a length of tubing, and urine from the bladder is allowed to drip in an uncontrolled fashion into the bag. When the bag becomes filled with urine, the attendant applies a clamp to the catheter or tubing, detaches the bag for emptying or replacement, and then re-attaches a bag. After a new or empty bag is attached, the clamp may be removed.

A number of disadvantages and difficulties are created with systems of the type described above. Clamps applied to the catheter or tubing often create less than perfect seals, and urine can leak out when the bag is being changed or emptied. Serious problems can occur if the clamp is left in place for too long of a time period, e.g. if the attendant becomes distracted or called away, by allowing the buildup of urine upstream from the clamp and in the bladder. Further, the bag is often difficult to detach and re-attach to the catheter or tubing to ensure no leakage occurs at that connection.

Additionally, many problems arise with the catheters used in such systems, particularly considering that the urine is allowed to flow in an essentially constant drip from the bladder, through the catheter and into the collection bag. In many cases, urine can pool and stagnate in the neck of the bladder when using a catheter causing struvite crystal encrustations. The pH of urine may change from a normal level to an ammonia state when pooled, resulting in precipitation and enlargement of struvite crystals on the indwelling catheter. Stagnation of urine may also create infections which can migrate to other areas of the body. Painful bladder spasms are another problem created by indwelling catheters, particularly during long periods of catheterization, which causes the bladder to cease its normal cycle of filling and flushing.

Little has been done to address the problems with prior urine collection systems, and other systems for collecting bodily fluids. Catheter-type devices have been proposed which employ siphoning, suction and various types of awkward manually externally controlled cycling apparatus and procedures. No systems are capable of permitting the bladder to fill to a normal level, void or washout all at one time, while minimizing the buildup of infectious contaminants or their entry into the system and allowing the convenient detachment of a filled collection bag from the system with a minimum of effort and no leakage.

### SUMMARY OF THE INVENTION

This invention is directed to a system for collecting bodily fluids, and particularly urine from the bladder of a patient, comprising a catheter inserted into the bladder, a low pressure magnetic valve coupled to the catheter, a collection bag, and, a quick-release coupler interposed between the magnetic valve and the collection bag.

Different types of collection bags may be employed in the system of this invention, including bags used when the patient is sleeping and mobile bags which may be attached to the leg or around the waist of a patient. Each bag has an inlet provided with a flap valve or the like to prevent the escape of fluid once it enters the bag, and a drain valve is located opposite the inlet to empty the contents of the bag.

The magnetic valve is a low pressure cyclic valve which is responsive to upstream fluid pressure in the system. The valve is held in the closed position by operation of magnetic force, the magnitude of which may be varied, and then moved to an open position when the pressure of the fluid contained in the bladder, catheter and tubing upstream from the valve reaches a selected level. As a result, urine within the bladder is voided or “washed out,” all at once, taking with it bacteria and other microbes which may otherwise accumulate in the area of the neck of the bladder. The magnetic valve of the system herein provides enhanced patient safety and comfort, and may help to prevent the bladder detrusor muscle from becoming flaccid or atonic during the period of catheterization.

The coupler of the system of this invention comprises an actuator housing coupled to the inlet of the collection bag, and a plunger housing coupled to tubing extending from the magnetic valve. Both of the housings are formed with a passageway for transmitting fluid exiting the magnetic valve to the collection bag. As the housings are connected together, a plunger located in the passageway of the plunger housing is moved from a closed position to an open position by an actuator mounted in the actuator housing in such a way that a seal is continuously maintained in the plunger housing to prevent the escape of fluids from its passageway. A quick-release locking mechanism maintains the two housings in a locked position when the plunger is in the open position, and may be easily activated to permit separation of the housings from one another when it is desired to empty or replace the collection bag.

In one presently preferred embodiment, fluid from the magnetic valve enters the inlet end of the passageway formed in the plunger housing. With the plunger in a closed position, a seal is created at the outlet end of the passageway to prevent leakage of fluid from the plunger housing. A second seal is mounted near the outlet end of the passageway which extends outwardly from the exterior surface of the plunger housing.

The actuator housing is movable relative to the plunger housing. An inner section of the actuator housing slides along the exterior surface of the plunger housing and engages the second seal protruding from the exterior surface of the plunger housing. A seal is created at that interface throughout substantially the entire period of contact between the two housings. The actuator is located downstream within the passageway of the actuator housing such that it does not contact the leading end of the plunger until after a seal is formed between the inner section of the actuator housing and the exterior surface of the plunger housing. Consequently, when the actuator does move the plunger to an open position, unseated from the outlet end of the passageway in the plunger housing, the two housings are sealed together preventing the escape of any fluids from the plunger housing except into the passageway of the actuator housing.

The locking mechanism is coupled to the exterior surface of the plunger housing and is effective to maintain the two housings connected to one another while the plunger is in the open position. A release button of the locking mechanism allows the two housings to be easily and quickly separated from one another, usually with one hand, so that the collection bag may be emptied or replaced. In the course of separating the two housings from one another, the plunger moves to the closed position prior to disengagement of the inner section of the actuator housing from the seal on the exterior surface of the plunger housing. This ensures that any fluid within the plunger housing is captured and maintained within its passageway before the two housings are separated from one another. As a result, there is no dripping of fluid from the plunger housing either when the two housings are connected to one another or when they are separated.

### BRIEF DESCRIPTION OF THE DRAWINGS

The structure, operation and advantages of the presently preferred embodiment of this invention will become further apparent upon consideration of the following description, taken in conjunction with the accompanying drawings, wherein:

FIG. 1 is a schematic view of the system of this invention with the catheter inserted in the bladder, and with a collection bag intended for use while a patient is sleeping;

FIG. 2 is a schematic view of an alternative collection bag for affixing to the leg of a patient;

FIG. 3 is a schematic view of an alternative collection bag for affixing around the waist of a patient;

FIG. 4 is perspective view of the plunger housing and actuator housing of the coupler of this invention, shown separated from one another;

FIG. 5 is an elevational view of the coupler depicted in FIG. 4, with the housings separated;

FIG. 6 is a cross sectional view taken generally along line 6-6 of FIG. 5; and

FIG. 7 is a cross sectional view of the coupler similar to FIG. 6, except with the two housings assembled.

### DETAILED DESCRIPTION OF THE INVENTION

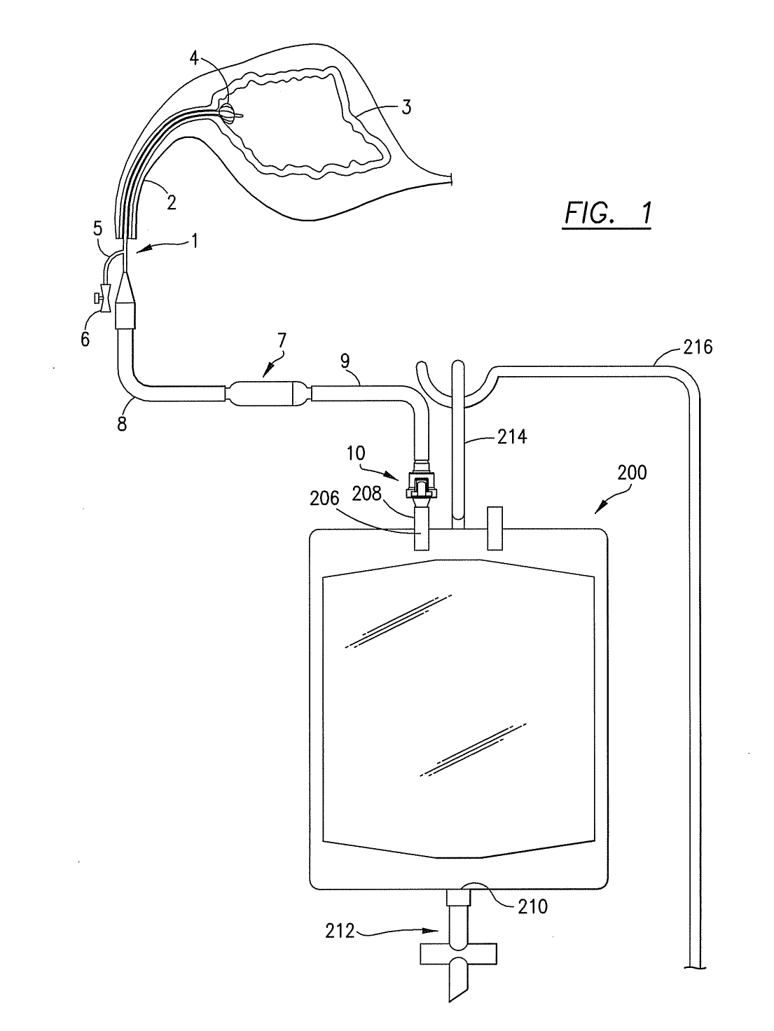

Referring initially to FIGS. 1-3, the fluid collection system of this invention generally comprises a catheter 1, a magnetic valve 7, a coupler 10 and a collection bag 200. The catheter 1 depicted in FIG. 1 is a conventional Foley catheter which is shown inserted through the urethra 2 into the bladder 3. The catheter 1 includes a balloon-tipped end 4, a balloon fill line 5 and a valve port 6. It should be understood that other indwelling catheters may be employed in the fluid collection system of this invention, including the balloonless catheter described in U.S. Pat. No. 6,855,126 to Flinchbaugh, the disclosure of which is incorporated by reference in its entirety herein.

The outlet of the catheter 1 is connected by tubing 8 to the inlet of magnetic valve 7. The magnetic valve 7 is preferably of the type described in U.S. Pat. No. 6,673,051 to Flinchbaugh, the disclosure of which is incorporated by reference in its entirety herein. As noted above, the magnetic valve 7 is held in a closed position by magnetic force. The valve 7 is movable to an open position in response to a selected level of fluid pressure, exerted by urine present in the bladder 3, catheter 1 and tubing 8 located upstream from the valve 7. When opened, the valve 7 allows urine to wash out of the bladder 3, i.e. flow out all at one time, along the flow path defined by the catheter 1, tubing 8 and the valve 7. Once the fluid pressure is released upstream from the valve 7, magnetic force returns the valve 7 to the closed position. The valve 7 may therefore be characterized as cyclic, whose frequency of operation is governed by how often the bladder fills with a quantity of urine sufficient to exert enough fluid pressure on the valve 7 to move it to the open position. As described in the '051 Flinchbaugh patent, the magnitude of magnetic force urging the valve 7 to the closed position may be varied to alter the level of fluid pressure required to open the valve 7, as desired.

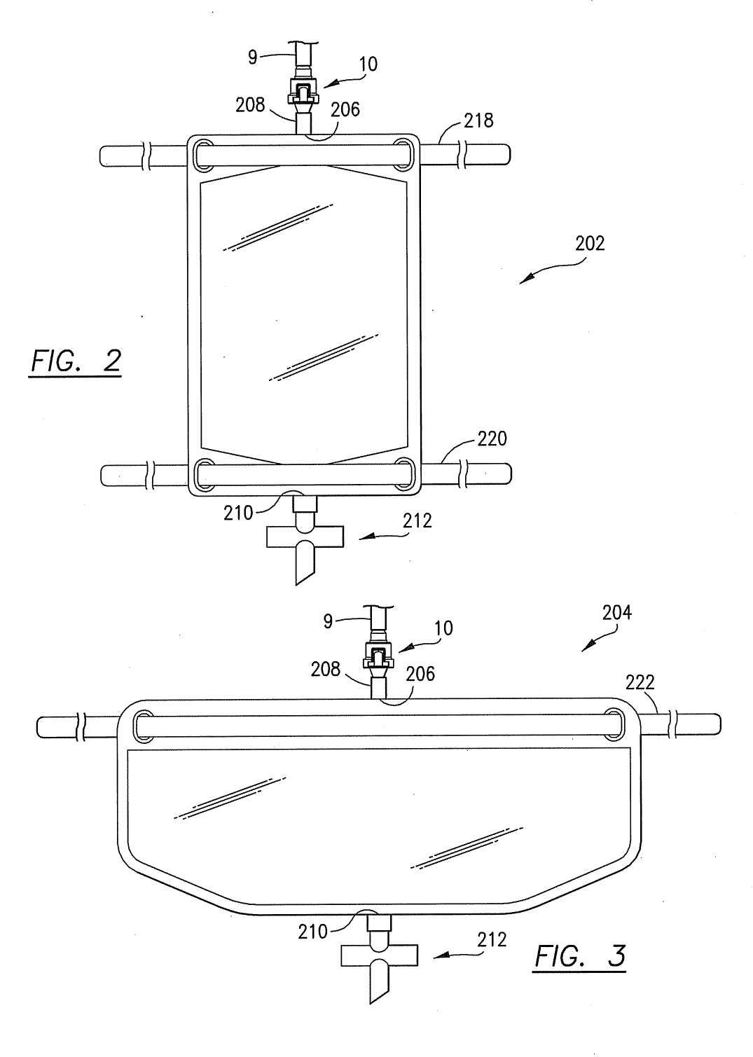

The outlet of valve 7 is coupled by tubing 9 to the inlet side of the coupler 10, which is described in detail below. As depicted in FIG. 1, the outlet side of the coupler 10 is connected to a collection bag 200. As alternatives to the collection bag 200, a leg bag 202 is shown in FIG. 2 and a “belly” bag 204 is illustrated in FIG. 3. Each of these bags 200, 202, 204 has a hollow interior, an inlet 206 coupled to an inlet tube 208 and an outlet 210 connected to a drain valve 212. A one-way valve or flap valve (not shown) is provided at the inlet 206 of each bag 200, 202 and 204 to prevent urine or other liquid from escaping out of the inlet 206 once it enters the interior thereof. The collection bag 200 shown in FIG. 1 is larger in size than the other two bags 202, 204 and is intended primarily for use during sleeping hours of the patient. It has a mounting clip 214 for attachment to a hook or other support 216 on the patient's bed, nightstand or the like. The leg bag 202 includes adjustable straps 218 and 220 at the top and bottom, respectively, preferably with hook and loop fastening elements at the ends of the straps 218, 220 to permit the bag 202 to be secured to the leg of a patient. The “belly” bag 204 is provided with a single mounting strap 222, having hook and loop fasteners at the free ends, which is sized to fit around and secure to the waist of a patient. When filled, each of these bags 200, 202, 204 may be separated from the coupler 10, as described below, and then emptied by operation of the drain valve 212.

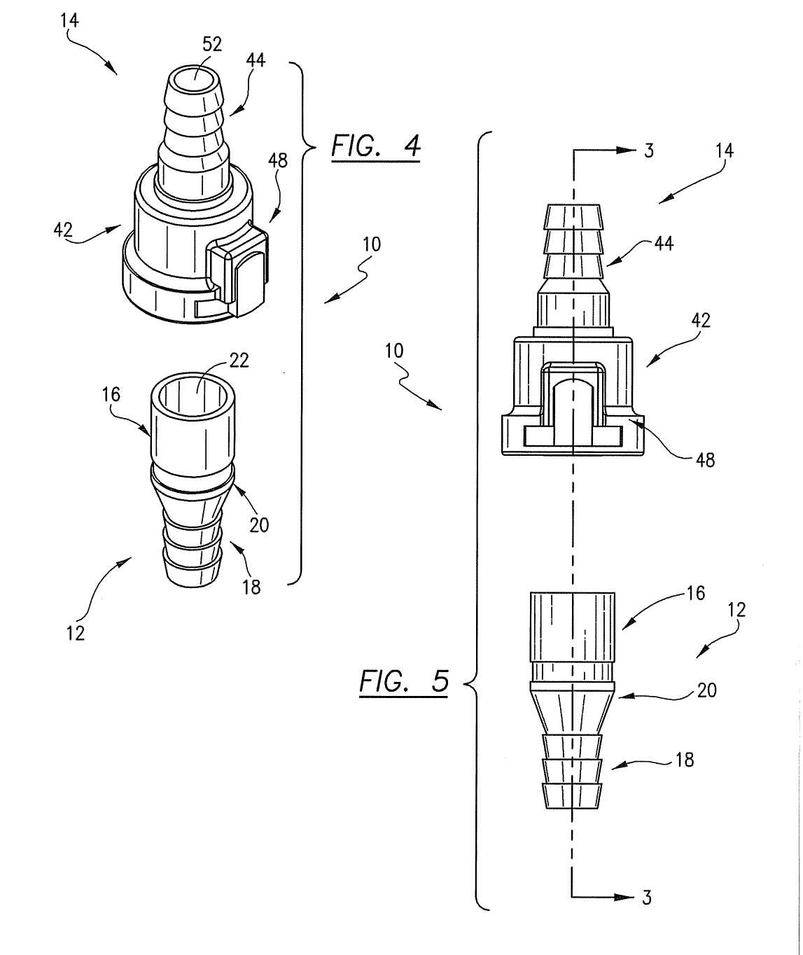

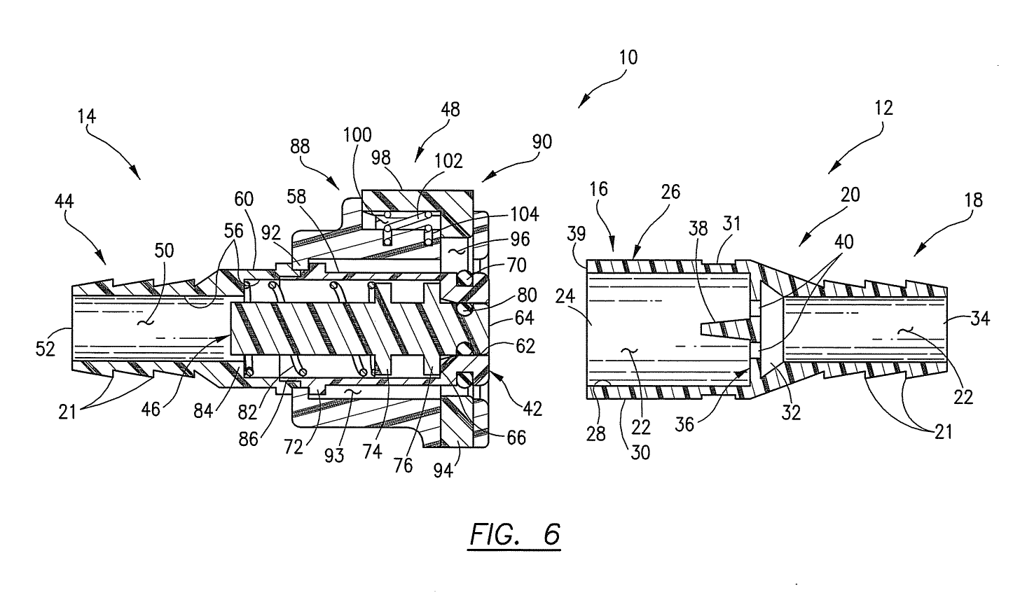

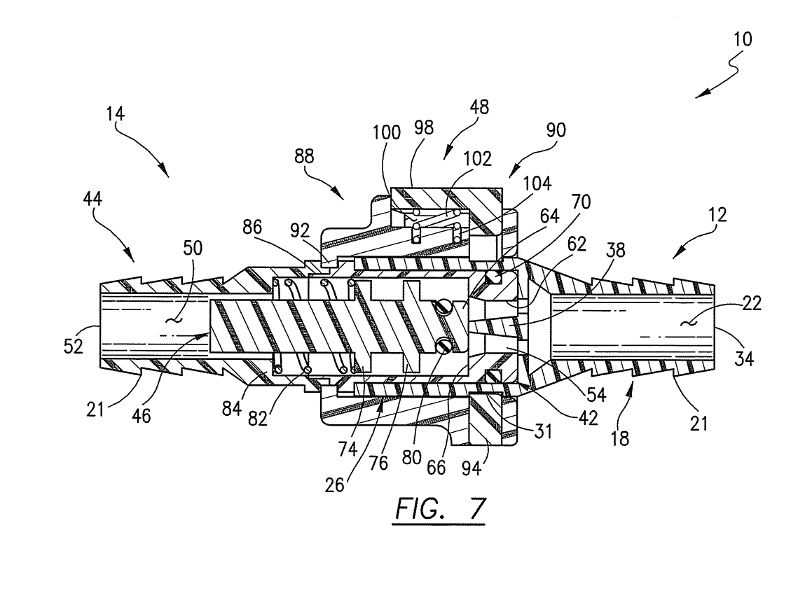

Referring now to FIGS. 4-7, the coupler 10 of this invention includes an actuator housing 12 and a plunger housing 14 which are releasably connected to one another. For purposes of the present discussion, the terms “inner” and “outer” refer to an orientation in the longitudinal direction as depicted in FIGS. 6 and 7, i.e. “inner” refers to the center of such Figs. and “outer” refers to a location toward the edges of the sheet on which such Figs. are drawn. The terms “interior” and “exterior” refer to radially spaced locations in FIGS. 3 and 4, it begin understood that “interior” surfaces are located toward the center of the housings 12 and 14, as shown. Additionally, the terms “inlet” and “outlet” refer to the direction of fluid flow through the coupler 10, e.g. from the left to the right in the orientation of the coupler 10 depicted in FIGS. 3 and 4.

The actuator housing 12 comprises an inner section 16, a longitudinally spaced outer section 18 and a tapered section 20 between the inner and outer sections 16, 18. The outer section 18 includes a number of longitudinally spaced, protruding barbs 21 which engage the inlet tube 208 of one of the collection bags 200, 202 or 204. The actuator housing 12 is formed with a passageway 22 having an inlet 24 at the inner section 16. The passageway 22 defines a generally cylindrical-shaped wall 26 with an interior surface 28 and an exterior surface 30. An annular recess 31 is formed in the inner section 16 which extends inwardly from the exterior surface 30 of the wall 26 toward the interior surface 28. The passageway 22 reduces in diameter through a transition area 32 along the tapered section 20 of actuator housing 12 to the outer section 18 where it terminates at an outlet 34. A disc 36 extends radially inwardly into the passageway 22 from the area where the inner and tapered sections 16, 20 meet. A plunger actuator 38 is mounted to the center of the disc 36, extending in a direction toward the inlet 24 of the passageway 22, and a number of bores 40 are formed in the disc 36 encircling the actuator 38. It should be noted, for purposes to become apparent below, that the actuator 38 is longitudinally spaced from the inlet 24 of the passageway 22 and the innermost edge 39 of the inner section 16 of the actuator housing 12.

The plunger housing 14 generally comprises an inner body 42, an outer body 44 and a plunger 46. A locking mechanism 48 is coupled to the plunger housing 14, as described in detail below. A passageway 50 extends through the plunger housing 14 having an inlet 52 at the outer body 44 and an outlet 54 at the inner body 42. See FIG. 4. The passageway 50 is defined by an interior surface 56 in the plunger housing 14. The inner body 42 has an exterior surface 58 spaced from the interior surface 56, and the exterior surface 60 of the outer body 44 is formed with barbs 21 as in the outer section 18 of actuator housing 12. The outer body 44 of the plunger housing 14 is connected to the tubing 9 extending from the outlet of magnetic valve 7.

As best seen in FIGS. 6 and 7, the inner body 42 tapers inwardly at the outlet 54 of the passageway 50 forming an elongated seat 62 which receives the leading end 64 of the plunger 46, when the plunger 46 in the closed position as described below in connection with a discussion of the operation of the coupler 10. The seat 62 terminates at a shoulder 66 upstream from the outlet 54 of the passageway 50. A groove extends radially inwardly from the exterior surface 58 of the inner body 42, adjacent to the outlet end of the inner body 42, and this groove mounts an O-ring 70 or similar seal. An annular ring 72 extends from the exterior surface 58 of the inner body 42 near its outer end.

The plunger 46 is formed with a pair of longitudinally spaced ribs 74 and 76, and a groove adjacent to its leading end 64. The groove mounts an O-ring 80, or a similar seal, in position to engage and seal against the seat 62 at the outlet of the inner body 42. With the plunger 46 in the closed position shown in FIG. 6, the rib 76 rests against the shoulder 66 of the inner body 42. A spring 82 extends along the exterior surface of the plunger 46 between the rib 74 and a shoulder 84 formed in the outlet end of the outer body 44 within the passageway 50. The longitudinal spacing between the rib 74 and shoulder 84 is chosen so that the spring 82 exerts sufficient force on the plunger 46 to maintain it in the closed position.

The plunger housing 14 is assembled by placing the spring 82 over the plunger 46 and then inserting the leading end 64 of the plunger 46 into the outlet end of the inner body 42 so that the O-ring 80 on the plunger 46 seals against the seat 62 of the inner body 42. The inner and outer bodies 42, 44 are then connected to one another, such as by adhesive or the like, along a joint 86 formed by mating annular surfaces at the ends of the bodies 42, 44.

The locking mechanism 48 comprises a lock collar 88 coupled to the inner body 42, and a release member 90 carried by the lock collar 88. The lock collar 88 extends around the inner body 42 of the plunger housing 14, and includes a radially inwardly projecting arm 92 affixed to the exterior surface 58 of the inner body 42 and the exterior surface 60 of the outer body 44 at the joint 86 between the two. An elongated, annular cavity 93 is formed between the lock collar 88 and exterior surface 58 of the inner body 42 which extends from the inner end of the inner body 42 toward the outer body 44. See FIG. 6. The release member 90 consists of an annular ring 94 carried within a slot 96 formed in the lock collar 88, and a button 98 extending generally perpendicularly to the ring 94. The button 98 slides within an opening 100 formed in the lock collar 88. A spring 102, located within the opening 100 and seated within an annular slot 104 in the lock collar 88, bears against the button 98 urging it radially outwardly as depicted in FIGS. 6 and 7. At least a portion of the annular ring 94 of the release member extends part way into the cavity 93 between the lock collar 88 and the exterior surface 58 of the inner body 42.

### System Operation

It is contemplated that the fluid collection system of this invention may be provided to the attendant at least partially assembled, although assembly can take place after catheterization. When the catheter 1 is positioned within the bladder 3, the magnetic valve 7 and tubing 8 may be quickly connected to the outlet of the catheter 1 to prevent any dripping. As discussed above, the magnetic valve 7 will not open and permit the passage of urine through it until a selected fluid pressure level is reached within the bladder 3, catheter 1 and tubing 8. The outer body 44 of the plunger housing 14 of coupler 10 is coupled to the tubing 9 from the outlet of valve 7, and the outer section 18 of the actuator housing 12 is coupled to the inlet tube 208 of one of the collection bags 200, 202 or 204. With the actuator housing 12 and plunger housing 14 of the coupler 10 separated from one another, the spring 82 urges the plunger 46 to the closed position in which the O-ring 80 at the leading end 64 of the plunger 46 contacts and seals against the seat 62 formed in the inner body 42 at the outlet 54 of the passageway 50. This prevents any leakage through the plunger housing 14.

In order to connect the two housings 12, 14 to one another, and move the plunger 46 to an open position, the wall 26 of the inner section 16 of the actuator housing 12 is inserted into the cavity 93 formed between the locking mechanism 48 and the exterior surface 58 of the inner body 42 of the plunger housing 14. After a minimum amount of travel along the cavity 93, the interior surface 28 of the wall 26 engages and seals against the O-ring 70 on the exterior surface 58 of the inner body 42 of the plunger housing 14. It is important to note that the plunger 46 is in the closed and sealed position at the time of contact between the wall 26 and O-ring 70. Continued longitudinal movement of the actuator housing 12 relative to the plunger housing 14 causes the actuator 38 to engage the leading end 64 of the plunger 46. Such movement overcomes the force exerted by the spring 82 tending to close the plunger 46, and moves the plunger 46 to an open position, spaced from the seat 62, as shown in FIG. 7. With the plunger 46 in the open position, the passageway 50 in the plunger housing 14 communicates with the passageway 22 in the actuator housing 12 thus forming a flow path through the coupler 10.

As best seen in FIG. 7, the wall 26 of the inner section 16 of actuator housing 12 continues along the cavity 93 until the annular recess 31 formed in the exterior surface 30 of the inner section 16 of actuator housing 12 aligns with the annular ring 94 of the locking mechanism 48. At that point, the spring 102 urges the ring 94 into the recess 31 thus locking the actuator housing 12 and plunger housing 14 in position relative to one another. See bottom of FIG. 7. In the locked position of the two housings 12 and 14, the plunger 46 is maintained in the open position.

With the housings 12 and 14 connected and locked together, a leak-proof flow path is provided from the valve 7 to a collection bag 200, 202 or 204. As discussed above, the valve 7 cyclically permits the bladder to washout, all at once, and discharges the urine through tubing 9 into the coupler 10. The urine passes through the coupler 10 into a collection bag 200, 202 or 206, with the O-ring 70 on the exterior surface 58 of the inner body 42 of plunger housing 14 preventing any leakage from the coupler 10.

When it is desired to disconnect the housings 12 and 14 from one another, to empty or replace a collection bag 200, 202 or 204, the release button 98 is pressed radially inwardly which causes the annular ring 94 to disengage the recess 31 in the wall 26 of the inner section 16 of actuator housing 12. This allows the inner section 16 of actuator housing 12 to be slid out of the cavity 93. As the actuator housing 12 is moved in a direction away from the plunger housing 14, the plunger 46 is urged in the same direction by the spring 82. As soon as the actuator 38 clears the outlet 54 of the passageway 50 in the plunger housing 14, the plunger 46 assumes its closed position with the O-ring 80 sealed against the seat 62 in the inner body 42 of plunger housing 14. At this juncture, the wall 26 of the inner section 16 of the actuator housing 12 remains sealed to the O-ring 70 on the exterior surface of the inner body 42 because of the longitudinal spacing between the actuator 38 and the innermost edge 39 of the inner section 16. This ensures that any fluid residing within the passageway 50 of the plunger housing 14 cannot escape as the housings 12 and 14 are disconnected from one another. The actuator housing 12 is then completely disengaged from the plunger housing 14 to permit a bag 200, 202 or 204 connected to the actuator housing 12 to be emptied or replaced. Fluid which entered the passageway 22 of the actuator housing 12 while the housings 12 and 14 were connected to one another would have already passed into a collection bag, 200, 202 or 204, which, as noted above, include a flap valve at the inlet 206 thereof to prevent fluid from exiting the bag 200, 202 or 204 at its inlet 206. After the collection bag is emptied or replaced, the housings 12 and 14 may be re-connected to one another as described above.

While the invention has been described with reference to a preferred embodiment, it should be understood by those skilled in the art that various changes may be made and equivalents substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. Apparatus for managing bodily fluids emitted from a patient, comprising:
an indwelling catheter which receives bodily fluids from a patient;
a flow control valve coupled to said indwelling catheter, said flow control valve being effective to permit the passage of fluid therethrough when the pressure of fluid upstream from said flow control valve reaches a selected level;
a collection bag;
a coupler connected between said flow control valve and said collection bag, said coupler comprising:
(i) an actuator housing having an inner section, an outer section and a first passageway extending through said inner and outer sections, an actuator being mounted to one of said inner and outer sections within said first passageway;
(ii) a plunger housing having a first end, a second end opposite said first end, a second passageway extending between said first and second ends, an interior surface defining said second passageway, an exterior surface spaced from said interior surface, and a plunger movable within said second passageway between an open position and a closed position, a leading end of said plunger being sealed against said interior surface of said passageway at said first end of said plunger housing when in said closed position;
(iii) said inner section of said actuator housing being movable in sealing engagement along said exterior surface of said plunger housing in a direction from said first end toward said second end of said plunger housing, said actuator of said actuator housing contacting said leading end of said plunger in the course of movement of said actuator housing relative to said plunger housing to move said plunger to said open position thereby interconnecting said first passageway of said actuator housing with said second passageway of said plunger housing; and
(iv) a locking mechanism coupled to said exterior surface of said plunger housing, said locking mechanism being effective to releasably connect said actuator housing and said plunger housing to one another with said plunger in said open position.

2. The apparatus of claim 1 in which said indwelling catheter is a Foley catheter.

3. The apparatus of claim 1 in which said flow control valve is a low pressure magnetic valve.

4. The apparatus of claim 1 in which said collection bag includes a hollow interior, an inlet and an outlet, said inlet being coupled to said first housing of said coupler, said outlet mounting a drain valve operative to drain fluid collected within said hollow interior.

5. The apparatus of claim 4 in which said collection bag further includes a mounting element adapted to connect to a bed.

6. The apparatus of claim 4 in which said collection bag includes at least one strap for mounting said bag to the leg of a patient.

7. The apparatus of claim 4 in which said collection bag further includes at least one strap for mounting said bag to the waist of a patient.

8. The apparatus of claim 1 in which said plunger housing of said coupler is formed with an inner body and an outer body which are connected to one another, said second passageway extending through said inner and outer bodies and having an inlet in said outer body and an outlet in said inner body.

9. The coupler of claim 8 in which said inner and outer bodies of said plunger housing of said coupler collectively form said interior and exterior surfaces of said plunger housing, said inner body mounting a seal which extends outwardly from said exterior surface in position to engage and create a seal with said inner section of said actuator housing.

10. The apparatus of claim 9 in which said seal mounted to said inner body of said plunger housing of said coupler is positioned relative to said leading end of said plunger so that a seal is created between said inner section of said actuator housing and said inner body of said plunger housing before said actuator of said actuator housing moves said plunger to said open position.

11. The apparatus of claim 1 in which said locking mechanism of said coupler comprises a lock collar coupled to said plunger housing, and a release member coupled to said lock collar, said release member including a ring movable within a slot formed in said lock collar and a release button operative to move said ring from a locked position to an unlocked position.

12. The apparatus of claim 11 in which a portion of said lock collar of said locking mechanism is spaced from said exterior surface of said plunger housing forming a cavity, said inner section of said actuator housing being received within said cavity in the course of movement of said actuator housing relative to said plunger housing.

13. The apparatus of claim 12 in which said ring of said release member of said coupler extends into said cavity.

14. The apparatus of claim 13 in which said inner section of said actuator housing of said coupler is formed with an annular recess, at least a portion of said ring of said release member extending into said annular recess of said inner section of said actuator housing to lock said actuator housing and said plunger housing together.

15. The apparatus of claim 14 in which said release button of said coupler is operative to disengage said at least a portion of said ring of said release member from said annular recess of said inner section of said actuator housing thus allowing said actuator housing to separate from said plunger housing.

16. The apparatus of claim 13 further including a spring acting on said release member of said coupler to urge said ring thereof into said cavity.
